(19) United States
(12) Patent Application Publication
(10) Pub. No.: US 2005/0181361 A1
(43) Pub. Date: Aug. 18, 2005

(54) Liver cancer prediction system for early detection and control method thereof

(76) Inventor: Dong-Kee Kim, Gyeonggi-do (KR)

Correspondence Address: LAHIVE & COCKFIELD, LLP., 28 STATE STREET, BOSTON, MA, 02109, US

(21) Appl. No.: 10/480,059

(22) PCT Filed: Sep. 30, 2003
(86) PCT No.: PCT/KR03/01997

(30) Foreign Application Priority Data
Oct. 1, 2002 (KR) 10-2002-0059894

(51) Int. Cl. C12Q 1/68; G06F 19/00; G01N 33/48; G01N 33/50

(52) U.S. Cl. 435/6; 702/19; 600/300

(57) ABSTRACT

The present invention relates to a liver cancer prediction system for early detection and control method thereof, which can perform hierarchical classification relating to a risk group for hepatocellular carcinoma, through an estimation of the incidence rate for the hepatocellular carcinoma and a relative risk of the incidence of hepatocellular carcinoma, both of which are found on an individual basis. General information on a patient, information depending on an ultrasonic test performed, clinical information including information on findings upon a first registration of a patient and information on findings upon a diagnosis of liver cancer, and information on a risk group, are stored in a database. A regression count which is an attributable ratio corresponding to each of risk factors is calculated based on the clinical information and risk group information stored in the database. An odds ratio of the incidence of liver cancer is measured by calculating risk probability of the incidence of liver cancer through a given operation process using the calculated regression count. It is thus possible to prevent the incidence of liver cancer per person depending on prediction of the incidence of liver cancer. Also hierarchical classification relating to a risk group for hepatocellular carcinomas is performed through the incidence rate for hepatocellular carcinoma and a relative risk of the incidence of liver cancer that are calculated on an individual basis. Therefore, a tailored model for prediction the incidence of liver cancer can be constructed.

### TECHNICAL FIELD

The present invention relates to a liver cancer prediction system for early detection and control method thereof. More particularly, the present invention relates to a liver cancer prediction system for early detection and control method thereof, which can predict liver cancer using an individual tailor-made model for predicting the incidence of liver cancer, in such a manner that hierarchical classification relating to a risk group for hepatocellular carcinoma is performed in consideration of an estimation of the incidence rate of hepatocellular carcinoma and a relative risk of the incidence of the hepatocellular carcinoma on a basis of information on risk factors, through the collection of prospective materials for analyzing a result of a long-term ultrasonic inspection.

### BACKGROUND ART

Liver cancer refers to a malignant tumor generated within the liver. It can be largely classified into hepatocellular carcinoma that is primarily generated within a hepatocyte, and metastatic liver cancer that is generated within extrahepatics and is then transferred to the inside of the liver. Liver cancer in this case means primary hepatocellular carcinoma. The hepatocellular carcinoma is one of the most common malignant tumors worldwide. The incidence rate of the hepatocellular carcinoma differs greatly from region to region. It is reported that hepatocellular carcinoma-prone regions are Africa and East Asia, where the incidence rate of hepatocellular carcinoma is 20 and above per 100,000 people. Whereas it is reported that the incidence rate of hepatocellular carcinoma is 10 or less per 100,000 people in U.S.A., North Europe, etc., with a relatively low incidence rate of this disease. Korea has a high incidence rate of hepatocellular carcinoma such as 30 per 100,000 male population and 7 per 100,000 female populations. Especially the incidence rate aged 40 to 60 is 74 in male and 15 in female, which is very high worldwide. Korea National Statistical Office reports that Korea has the second highest death rate in liver cancer next to Africa. According to the report on cancer death rate published by Korea National Statistical Office in 1996, about 10,000 persons died of this disease in a year, which shows a liver cancer death rate of 21.4%. This ratio is the second highest cancer after gastric cancer. The Office reports that the death rate of liver cancer in the forties to fifties is even higher than that of gastric cancer.

In order to prevent liver cancer, it is required that we must exactly know the incidence carcinogenesis of liver cancer. If there are medicines for completely hindering carcinogenesis, it will be possible to easily prevent cancer. In recent years, an effort to prevent cancer with medicines has been actively made. In terms of liver cancer, however, significant advancements have not yet been reported so far. Although there has been proposed a method of administrating a medicine that changes aflatoxin within the body to non-carcinogens in some regions where people are severely exposed to aflatoxin, it is still only in a research stage. Therefore, even if it is uncertain to know carcinogenesis, the best alternative prevention method available has to be driven. The most efficient method to prevent a liver caner is to remove or avoid risk factors for hepatocellular carcinoma.

The most widely used inspection methods for early detection of hepatocellular carcinoma are a liver ultrasonic inspection method and a serum alpha-feto protein level checking method. Computerized tomography (CT) is more accurate to detect the incidence of cancer than the ultrasonic inspection method, but it is impractical for a screening test due to the inconvenience and high cost. Meanwhile, the ultrasonic inspection method is easy to use and has a detection sensitiveness of about 75% or more for even a tumor of a size less than 3 cm. Accordingly, the ultrasonic inspection method has been widely used for the screening test for early detection of liver cancer.

However, there is no method to analyze the long term individual ultrasonic inspection for prediction of the incidence of liver cancer. A patient has to suffer from an inconvenience of receiving diagnosis of liver cancer through the ultrasonic inspection method every time.

### DISCLOSURE OF INVENTION

Accordingly, the present invention has been made in view of the above problems. The present invention provides a liver cancer prediction system for early detection and control method thereof, which can perform hierarchical classification relating to a risk group for hepatocellular carcinoma, through an estimation of the incidence rate for the hepatocellular carcinoma and a relative risk of the incidence of hepatocellular carcinoma, both of which are found on an individual basis.

To achieve the above objects, according to one aspect of the present invention, there is provided a liver cancer prediction system for early detection, including a controller for controlling the entire operation of the system; a display unit for displaying information and a graphic user interface depending on the operation of the system under the control of the controller; an input unit for inputting initial set values, selecting a given menu based on the information displayed on the display unit and inputting information corresponding to the selected menu; a plurality of databases for storing general information on a patient, information depending on an ultrasonic test performed, clinical information including information on findings upon a first registration of a patient and information on findings upon a diagnosis of liver cancer, and information on a risk group; a regression counter for calculating a regression count which is an attributable ratio corresponding to each of risk factors based on the clinical information and risk group information stored in the database; and an odds ratio measurement unit for measuring an odds ratio of the incidence of liver cancer by calculating risk probability of the incidence of liver cancer through a given operation process using the regression count calculated in the regression counter.

According to another aspect of the present invention, there is also provided a method of controlling liver cancer prediction system including a controller for controlling the entire operation of the system; a display unit for displaying information depending on the operation of the system and a graphic user interface under the control of the controller; an input unit for inputting initial set values, selecting a given menu according to information displayed on the display unit and inputting information corresponding to the selected menu; a plurality of databases for storing general information on a patient, information depending on an ultrasonic operation performed, clinical information including information on findings upon a first registration of a patient and information on findings upon a diagnosis of liver cancer, and information on a risk group; a regression counter for calculating a regression count which is an attributable ratio corresponding to each of risk factors based on clinical information and risk group information stored in the database; and an odds ratio measurement unit for measuring an odds ratio of the incidence of liver cancer by calculating risk probability of the incidence of liver cancer through a given operation process using the regression count calculated in the regression counter, comprising: a patient information-managing step of displaying, on a display unit, a given menu wherein general information on a patient can be written, and storing information inputted through the input unit in the database; an ultrasonic information-managing step of displaying, on the display unit, a corresponding menu wherein information depending on an ultrasonic test performed can be written, and storing information inputted through the input unit in the database; a clinical information-managing step of displaying, on the display unit, a given menu wherein information on findings upon a first registration of a patient and information on finding upon a diagnosis of liver cancer can be written, and storing information inputted through the input unit in the database; a risk group-assigning step of displaying, on the display unit, a menu of a given format wherein additional risk groups can be assigned after the clinical information-managing step, and storing s risk group assigned according to information inputted through the input unit in the database; and an odds ratio measurement step of measuring an odds ratio of the incidence of liver cancer, by calculating probability of the incidence of liver cancer on the basis of clinical information stored in the clinical information-managing step and the risk group assigned in the risk group-assigning step.

### BRIEF DESCRIPTION OF DRAWINGS

Further objects and advantages of the invention can be more fully understood from the following detailed description taken in conjunction with the accompanying drawings in which:

FIG. 1 is a diagram showing the connection of a prediction system based on the embodiment of the present invention,

FIG. 2 is a block diagram illustrating the construction of a prediction system based on the embodiment of the present invention;

FIG. 3 is a block diagram illustrating the construction of a web server based on the embodiment of the present invention;

FIG. 4 is a block diagram illustrating the construction of a prediction server based on the embodiment of the present invention;

FIG. 5 is a block diagram illustrating the entire configuration of a graphic user interface (GUI) of the prediction system based on the embodiment of the present invention;

FIG. 6 is an exemplary view showing a GUI of an initial main menu based on the present invention;

FIG. 7 is an exemplary view showing a GUI of a patient information-managing menu based on the present invention;

FIG. 8 is an exemplary view showing a GUI of an ultrasonic information-managing menu based on the present invention;

FIG. 9 is a table showing a list of information stored in a database based on the present invention;

FIG. 10 is an exemplary view showing a GUI of a clinical information-managing menu based on the present invention;

FIG. 11 is an exemplary view showing a GUI of a risk group-assigning menu based on the present invention;

FIG. 12 is an exemplary view showing a GUI of a core risk factor menu based on the present invention;

FIG. 13 is an exemplary view showing a GUI of an extended risk factor menu based on the present invention;

FIG. 14 is an exemplary view showing a list of risk factors for measuring an odds ratio based on the present invention;

FIG. 15 is the entire flowchart that explains a process of predicting liver cancer based on the present invention; and

FIG. 16 is a flowchart that explains a process of a result notification based on the present invention.

### BEST MODE FOR CARRYING OUT THE INVENTION

The present invention will now be described in detail with reference to the accompanying drawings.

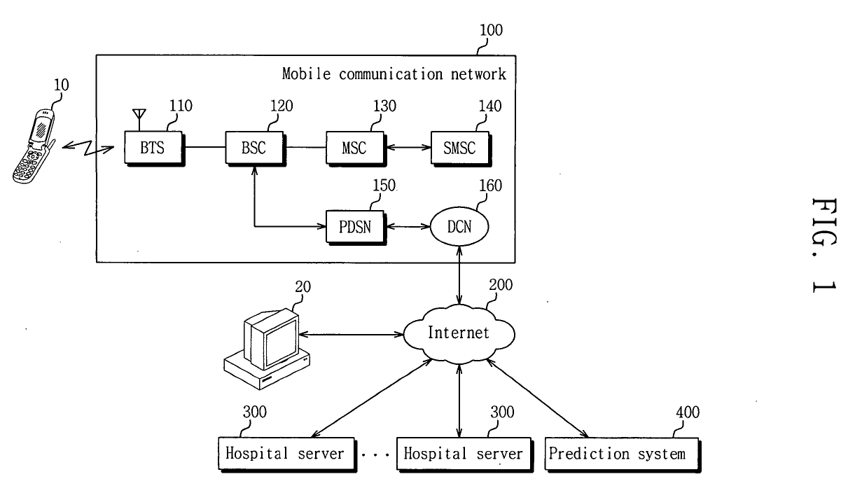

FIG. 1 is a diagram showing the connection of the prediction system based on the embodiment of the present invention.

Referring to FIG. 1, the prediction system 400 of the present invention is connected to a user computer terminal 20 and a plurality of hospital servers 300 through the Internet 200. The system 400 can transmit short messages to a user mobile communication terminal 10 via a mobile communication network 100.

The mobile communication network includes a base transceiver system (hereinafter, referred to as “BTS”) 110 that communicates by wireless with a user's mobile communication terminal 10, a base station controller (hereinafter, referred to as “BSC”) 120 that controls the BTS 110, a mobile switching center (hereinafter, referred to as “MSC”) 130 connected to the BSC 120 for performing call switching, and a short message servicing center (hereinafter, referred to as “SMSC”) 140 connected to the MSC 130 to control the short message.

Also a packet data-servicing node (hereinafter, referred to as “PDSN”) 150 for servicing packet data is connected to the BSC 120 of the mobile communication network 100. The PDSN 150 can provide an Internet 200 connection to the mobile communication terminal 10 via a data core network (hereinafter, referred to as “DCN”) 160.

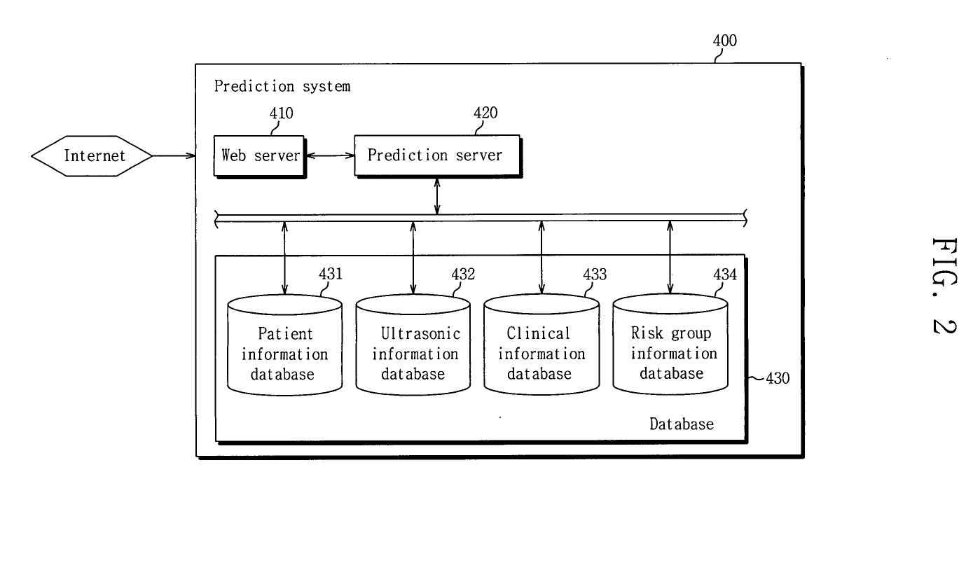

FIG. 2 is a block diagram illustrating the construction of a prediction system based on the embodiment of the present invention.

Referring to FIG. 2, the prediction system 400 includes a web server 410 that provides a web service to the user computer terminal 20 through the Internet 200, a prediction server 420 which detects a liver cancer, and a database 430 which stores/manages data.

The web server 410 provides a web service for predicting liver cancer to the connected user computer terminal 20 through the Internet 200. The web server 410 provides data relating to liver cancer prediction in the form of a short message or E-mail to the user terminal 10 or 20, in cooperation with the prediction server 420.

The prediction server 420 controls the plurality of the hospital servers 300 to collect/manage patient information through the Internet 200.

Further, the database 430 includes a patient information database 431 for storing/managing information on a patient, an ultrasonic information database 432 for storing/managing ultrasonic information, a clinical information database 433 for storing/managing clinical information on a patient, and a risk group information database 434 for storing/managing information on a risk group.

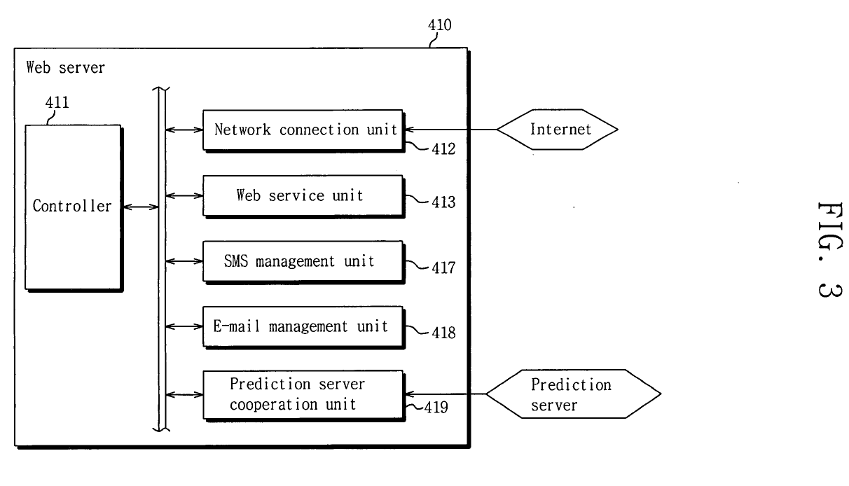

FIG. 3 is a block diagram illustrating the construction of the web server 410 based on the embodiment of the present invention.

Referring to FIG. 3, the web server 410 includes a controller 411 for controlling the entire operation, a network connection unit 412 for connecting to the Internet 200, a web service unit 413 for providing the user terminal 10 or 20 with a web service through the Internet 200, an short message servicing (hereinafter, referred to as “SMS”) management unit 417 for generating a short message and providing it to a user's mobile communication terminal 10 through the mobile communication network 100, an E-mail management unit 418 for generating E-mail and transmitting it to an E-mail account of a user, and a prediction server cooperation unit 419 that cooperates with the prediction server 420.

The web server 410 which was constructed by the present invention provides a web service for predicting liver cancer to the user terminal 10 or 20 that is connected to the Internet 200 through the web service unit 413. In this case, a user's mobile communication terminal 10 is connected to the Internet 200 in a wireless manner via the mobile communication network. A user computer terminal 20 is connected to the Internet through a wired network.

The web server 410 receives the result about the liver cancer prediction from the prediction server 420 via the prediction server cooperation unit 419, and generates a short message containing the result through the SMS management unit 417. Furthermore, the web server 410 transmits the generated short message to a mobile communication terminal 10 of the attending physician of a corresponding patient who is previously registered through the mobile communication network 100.

In addition, the web server 410 generates E-mail containing the result through the E-mail management unit 418, and transmits the generated E-mail to an E-mail account of the attending physician of the corresponding patient.

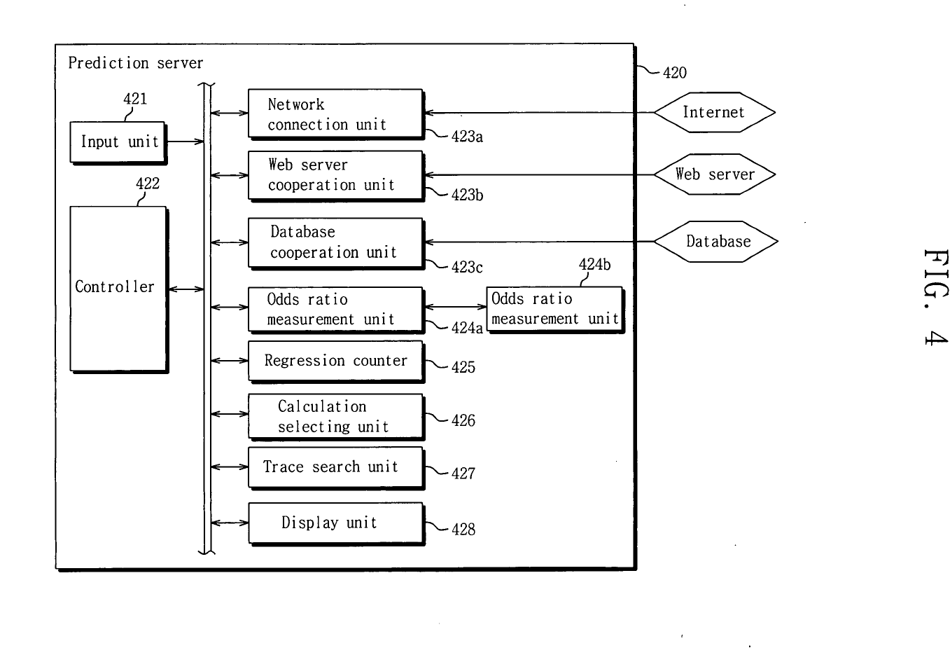

FIG. 4 is a block diagram illustrating the construction of the prediction server 420 for early detection of liver cancer based on the embodiment of the present invention.

Referring to FIG. 4, the prediction server 420 includes a controller 422 for controlling the entire operation, a display unit 428 for performing a window display so that information depending on the operation of the controller 422 can be visually viewed, an input unit 421 for inputting given data or commands based on the information displayed on the display unit 428, a network connection unit 423a for connecting the prediction server 420 and the Internet, a web server cooperation unit 423b for exchanging data with the web server 410, and a database cooperation unit 423c for accessing the database 430 to store/manage data.

The prediction server 420 further has a regression counter 425 for calculating a regression count which is an attributable ratio corresponding to each risk factor based on the clinical information and the risk group information stored in the database 430, and an odds ratio measurement unit 424a for measuring an odds ratio of the occurrence of a liver cancer by calculating incidence probability of liver cancer through a given operation process using the regression count calculated in the regression counter 425. In the above, the odds ratio measurement unit 424a is connected to an odds ratio storage unit 424b for storing the odds ratio and risk probability that are previously produced.

The prediction server 420 further has a calculation-selecting unit 426 for selecting whether to calculate risk probability using core risk factors or using an extended model. The prediction server 420 also includes a trace search unit 427 for searching a trace monitoring item from risk group-assigning materials that are previously stored in the database when the extended risk factor is selected.

The prediction server 420 transmits the result on the calculated risk probability to the web server 410 so that it can be transmitted to the attending physician of a corresponding patient in the form of a short message or E-mail.

An operational process based on the present invention will now be described with reference to the drawings.

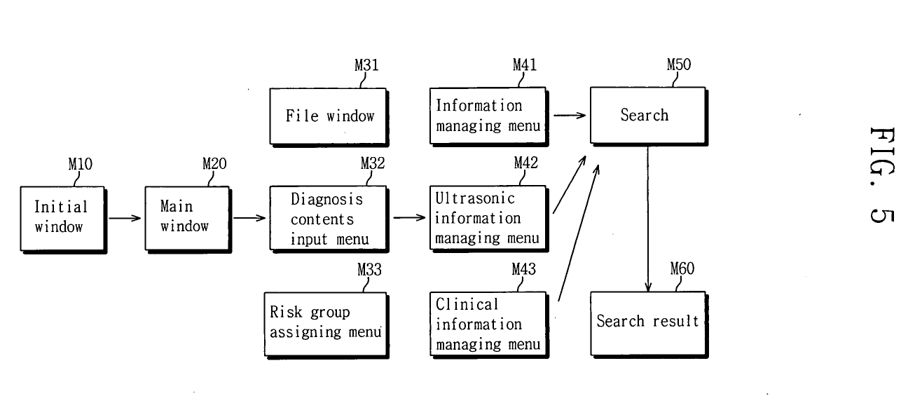

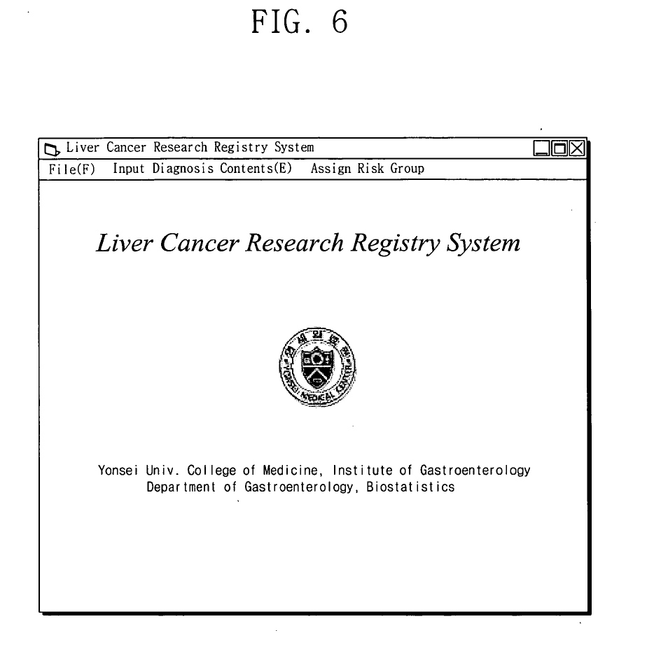

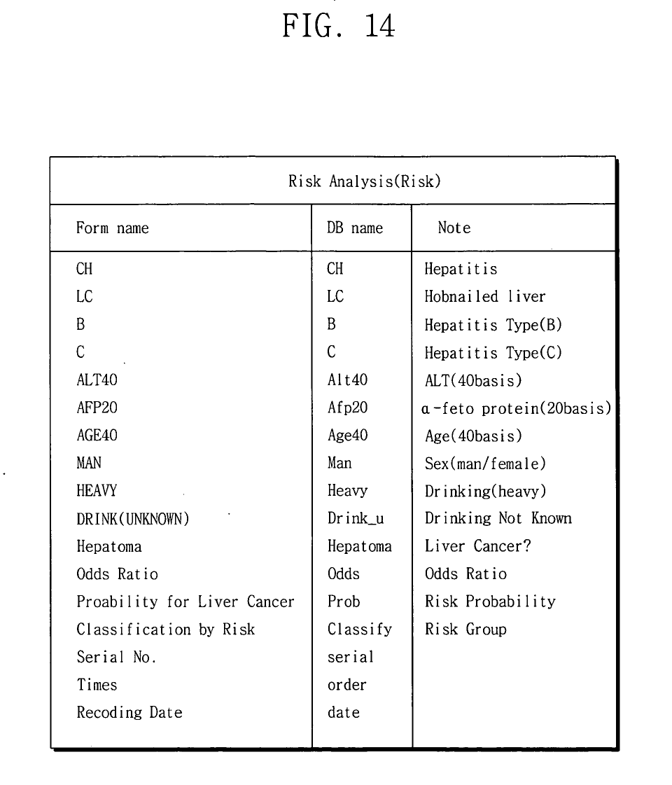

FIG. 5 is a block diagram illustrating the entire configuration of a graphic user interface (GUI) of the prediction system based on the embodiment of the present invention. FIG. 6 and FIG. 14 are exemplary views showing applications of the graphic user interface shown in FIG. 5 and the configuration of a data table.

Referring to FIG. 5, when the prediction system of the present invention is executed, an initial window M10 is displayed and then a main window M20 as shown in FIG. 6 is displayed on the display unit 428 of the prediction server 420. The GUI of the main window has a file window M31 that supports storage/conversion/deletion of data, database conversion, etc., a diagnosis contents input menu M32 for executing patient data input, ultrasonic data input and clinical data input, and a risk group-assigning menu M33 for assigning a risk group. Based upon the selection by the input unit 421, the controller 422 of the prediction server 420 displays a GUI for inputting corresponding data on the display unit 428 and receives the input data through the input unit 421.

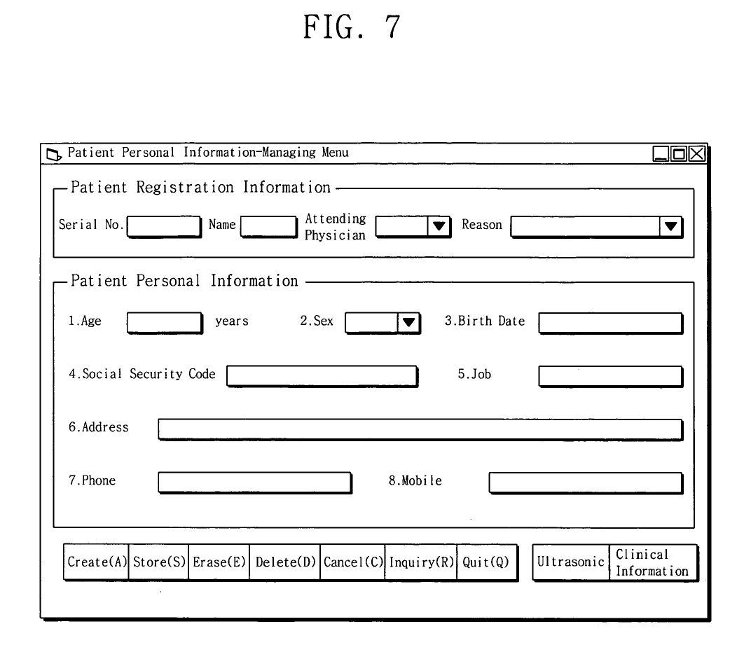

Meanwhile, if a command to select the diagnosis contents input menu M32 is inputted through the input unit 421, the controller 422 controls the display unit 428 to display GUIs of a patient information-managing menu M41, an ultrasonic information-managing menu M42 and a clinical information-managing menu M43. The details are as follows. If a command to select the patient information-managing menu M41 is inputted through the input unit 421, the controller 422 controls the display unit 428 to display a GUI as shown in FIG. 7. Accordingly, a user can create, store, modify, delete, cancel, inquire registration information and personal information on a patient, or finish the menu.

If registration information or personal information on a patient is inputted through the graphic user interface and the input unit 421, the controller 422 of the prediction server 420 controls the database cooperation unit 423c to create, store, modify, delete, cancel, or inquire the data in the patient information database 431 of the database 430.

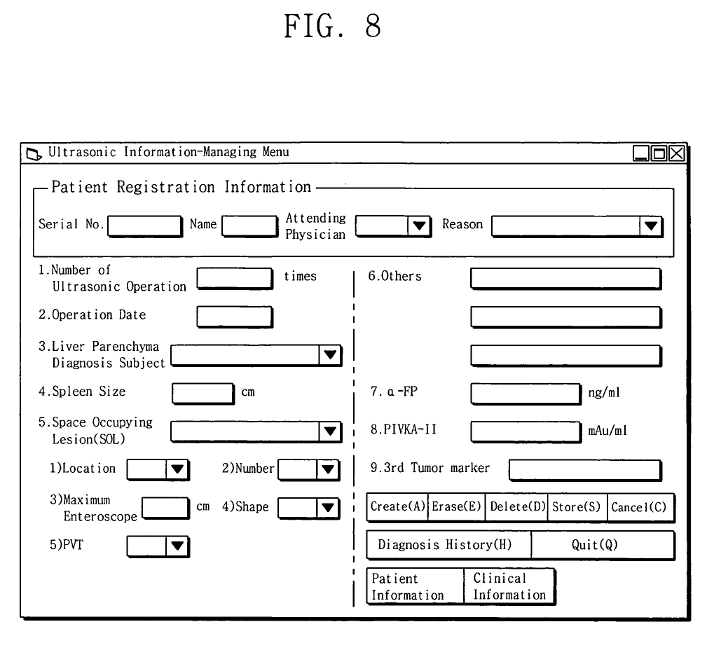

Meanwhile, if a command to select the ultrasonic information-managing menu M42 is inputted through the input unit 421, the controller 422 controls the display unit 428 to display a GUI as shown in FIG. 8. The ultrasonic information-managing menu M42 serves to input information depending on an ultrasonic test. If a menu execution command is inputted, the controller 422 first controls the display unit 428 to display the GUI. The controller 422 then controls the database cooperation unit 423c to request/receive registration information on a corresponding patient that is stored in the patient information database 431, and then makes the received data to be displayed at a corresponding item of the GUI.

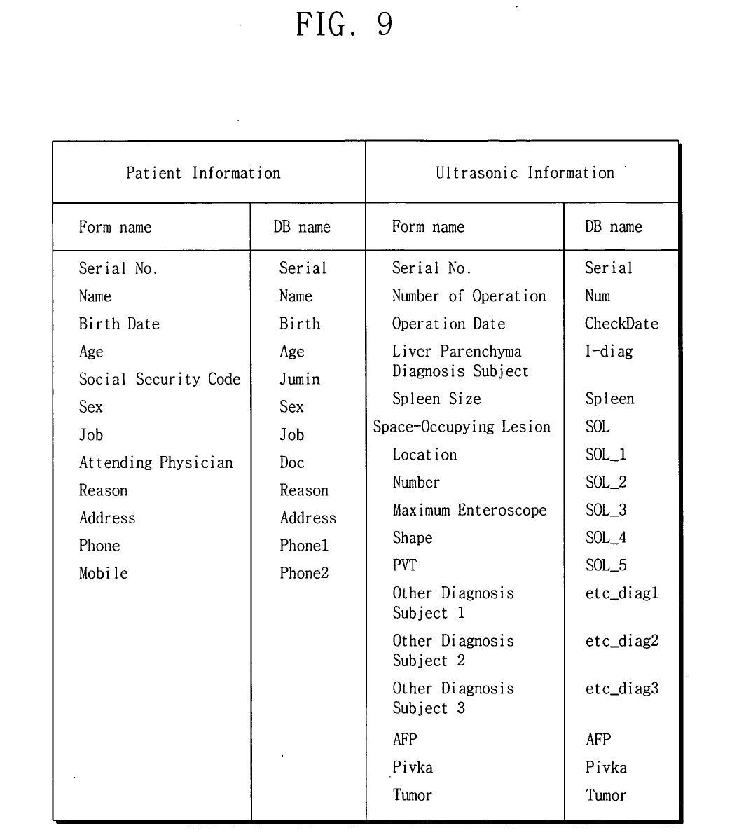

The controller 422 controls the database cooperation unit 423c to store/manage ultrasonic information inputted through the input unit 421 in the ultrasonic information database 432. At this time, the ultrasonic information is controlled to be matched to patient information and then stored in the database 432. An embodiment of each of parameters of patient information and ultrasonic information is shown in FIG. 9.

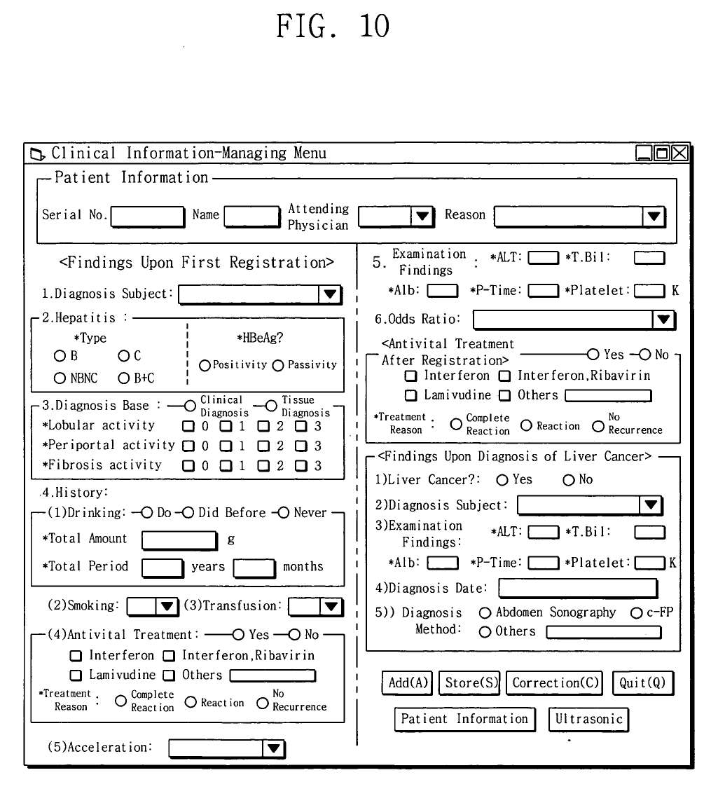

Moreover, if a command to select the clinical information-managing menu M43 is inputted through the input unit 421, the controller 422 controls the display unit 428 to display a GUI as shown in FIG. 10.

The clinical information consists of parameters such as a diagnosis subject, hepatitis, diagnosis basis, a case history, examination opinions, an odds ratio, and the like. If given data are inputted through the input unit 421, the controller 422 controls the database cooperation unit 423c to store/manage the inputted data in a corresponding database.

In this case, if data for patient information are inputted through the input unit 421, the controller 422 controls the database cooperation unit 423c to search corresponding patient information through the patient information database 431. If desired patient information is found, the controller 422 controls the searched patient information to be displayed at a corresponding item through the graphic user interface. Furthermore, the controller 422 searches clinical information coincident with corresponding patient information through the clinical information database 433. The controller 422 then controls the searched clinical information to be displayed at a corresponding item of the graphic user interface.

Meanwhile, if desired patient information is not found, the controller 422 determines that the data are new patient information. Accordingly, the controller 422 controls the display unit 428 to display a message indicating that “there is no matching information as a result of the search” and a message that prompts a user to input information through the input unit 421, for example, “There exists no such patient information. Please input the new patient information”. Also the controller 422 controls the database cooperation unit 423c to store information inputted via the input unit 421 in the database 430.

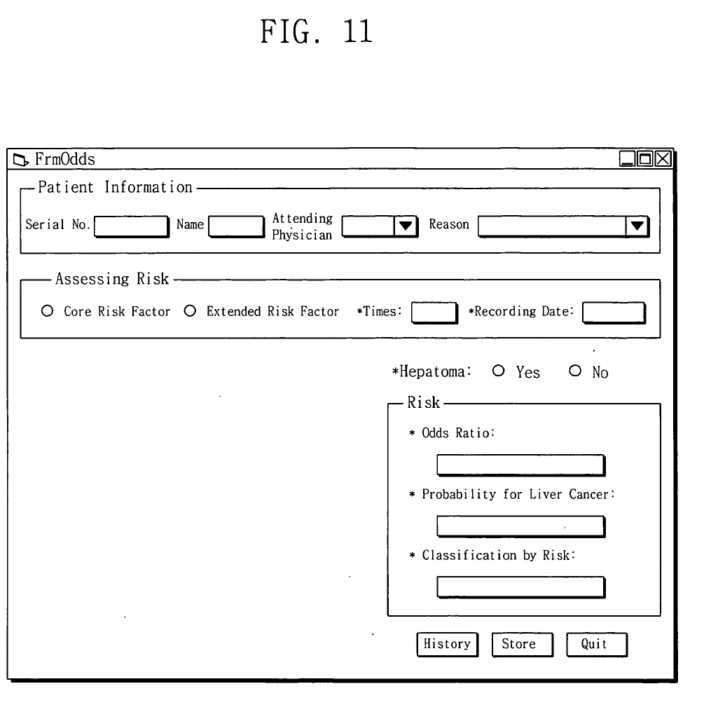

Meanwhile, if a command for the risk group-assigning menu M33 is inputted through the input unit 421, the controller 422 controls the display unit 428 to display a GUI as shown in FIG. 11.

Then, the controller 422 searches patient information from the patient information database 431 where the data are inputted through the input unit 421. The controller 422 controls the searched patient information to be displayed through the GUI.

Furthermore, the controller 422 searches information on a risk group coincident with corresponding patient information from the risk group database 434. The controller 422 then controls information on the searched risk group to be displayed at a corresponding item of the GUI.

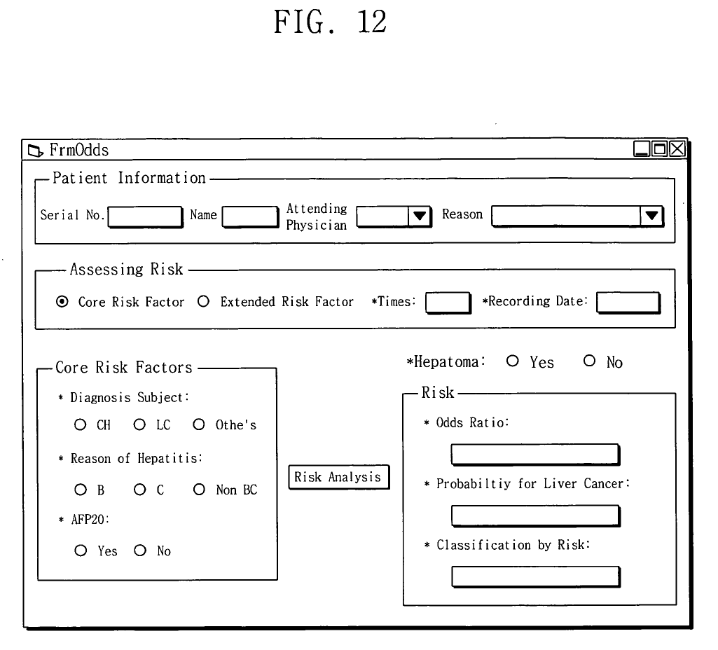

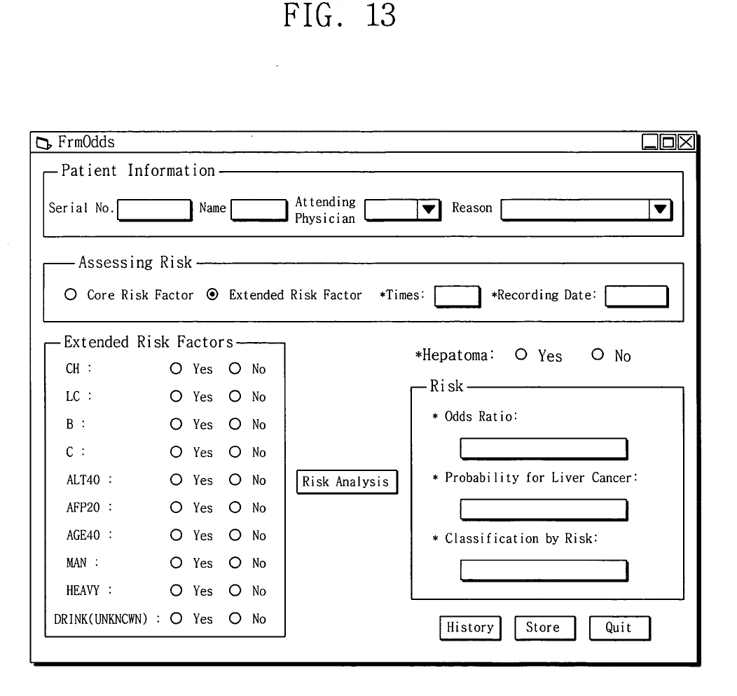

In addition, when the controller 422 calculate the risk probability of the risk group the controller 422 can select whether the odds ratio will be calculated using a core risk factor or an extended risk factor in accordance with a command inputted through the input unit 421. At this time, if a command to select the core risk factor is inputted via the input unit 421, the controller 422 displays a graphic user interface as shown in FIG. 12. If a command to select the extended risk factor is inputted, the controller 422 controls the display unit 428 to display a GUI as shown in FIG. 13.

In the event that the command for the extended risk factor is inputted, the controller 422 determines whether a “history” command is inputted. If the history command is inputted through the input unit 421, the controller 422 controls the database cooperation unit 423c by the trace search unit 427 and searches a trace monitoring item of a corresponding patient from information on a risk group that is stored/managed in the risk group information database 434. Further, the controller 422 controls the display unit 428 to display the searched information.

The risk factors based on the risk group information may consist of hepatitis, liver cirrhosis, hepatitis furuncle, ALT, α-FP (feto protein), age, sex (man/female), tolerance level to alcohol, whether drinking history is known or not, probability for liver cancer, odds ratio, risk probability and a risk group, as shown in FIG. 14.

Meanwhile, if patient information, ultrasonic information, clinical information and information on the risk group are each inputted according to the above procedure, the controller 422 calculates the odds ratio based on the inputted information. There are two methods to calculate the odds ratio. The controller 422 may use a method of calculating an odds ratio using three kinds of core risk factors such as the diagnosis subject, the cause of hepatitis and alpha fetoprotein (hereinafter, referred to as “AFP”), or a method of calculating an odds ratio using an extended risk factors where other control factors including the three kinds of the core risk factors are taken into consideration. The controller 422 selects a calculation method through the calculation-selecting unit 426 based on the selection of the core risk factors or the extended risk factors in the above-mentioned procedure.

When a method of calculating the odds ratio is decided, the controller 422 calculates a regression count (attributable ratio) corresponding to each risk factor shown in FIG. 14 through the regression counter 425. In other words, the controller 422 defines three kinds of risk factors (diagnosis subject, the cause of hepatitis, and AFP) for setting a logistic regression model as numerical parameters and then calculates the attributable ratio of each of the three kinds of the risk factors in the regression counter 425.

Thereafter, the odds ratio measurement unit 424a calculates risk probability through the logistic regression model using the calculated regression count under the control of the controller 422. That is, the odds ratio measurement unit 424a calculates a new odds ratio and a risk probability value by inserting the regression count for each of the risk factors calculated in the regression counter 425 and a statistical prediction model depending on the risk probability and odds ratio of the incidence of liver cancer that are previously produced into the logistic regression calculation formula. The controller 422 controls the display unit 428 to display the new calculated risk probability value.

The odds ratio measurement unit 424a updates/stores information on the calculated risk probability in the odds ratio storage unit 424b. At this time, the controller 422 transmits the result to the web server 410 via the web server cooperation unit 423b. The web server 410 can generate a short message containing the result through the SMS management unit 417, and then transmit the short message to the mobile communication terminal 10 of the attending physician that is previously registered, through the mobile communication network 100. In addition, the web server 410 can generate E-mail containing the result through the E-mail management unit 418 and then transmit the generated E-mail to an E-mail account of the attending physician that is previously registered, through the Internet 200.

The control method as described above will now be described with reference to a flowchart.

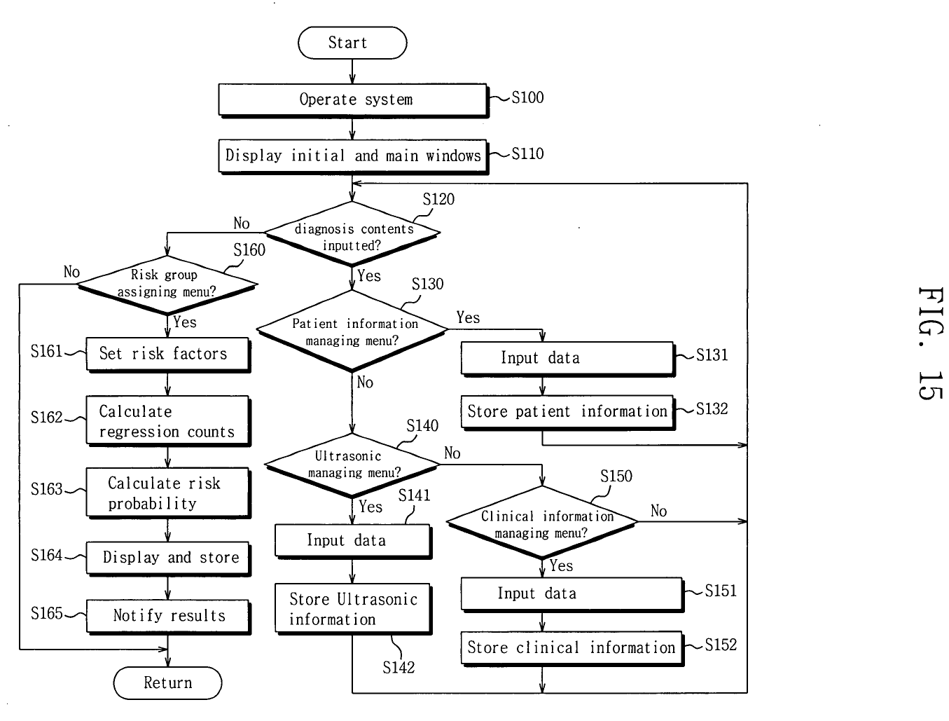

FIG. 15 is the entire flowchart for explaining a process of prediction liver cancer based on the present invention.

Referring to FIG. 15, when a system is driven (S100), the controller 422 of the prediction server 420 controls the display unit 428 to display initial and main windows (S110). The controller 422 then determines whether a command inputted through the input unit 421 is a command to input diagnosis contents (S120) or not.

If the command is a command to input diagnosis contents, the controller 422 determines whether a command for the patient information-managing menu can be inputted through the display unit 422 (S130). When the command for the patient information-managing menu can be inputted, the controller 422 outputs a message that prompts a user to input data on the display unit 428. The controller 422 then stores the data (S131) inputted through the input unit 421 in the patient information database 431 (S132).

If the command is not for the patient information-managing menu in step S130, the controller 422 determines whether the command is for the ultrasonic information-managing menu (S140). If the command is for the ultrasonic information-managing menu in step S140, the controller 422 outputs a message that prompts a user to input data on the display unit 428. The controller 422 then stores the data (S141) inputted through the input unit 421 in the ultrasonic information database 432 (S142).

If the command is not for the ultrasonic information-managing menu in step S140, the controller 422 determines whether the command is for a clinical information-managing menu (S150). If the command is for the clinical information-managing menu in step S150, the controller 422 outputs a message that prompts a user to input data on the display unit 428. The controller 422 then stores the data (S151) inputted through the input unit 421 in the clinical information database 433 (S152).

Meanwhile, if it is determined that the command is not the command instructing to input diagnosis contents in step S120, the controller 422 determines whether a command for the risk group-assigning menu is inputted (S160). If the command for the risk group-assigning menu is inputted, the controller 422 assigns a core risk factor or an extended risk factor based on information inputted through the input unit 421 (S161). The regression counter 425 then calculates a regression count that is assigned under the control of the controller 422 (S162).

When the regression count is calculated in step S162, the odds ratio measurement unit 424a uses the regression count to calculate an odds ratio under the control of the controller 422 (S163). The controller 422 controls the display unit 428 to display the calculated odds ratio. At the same time, the controller 422 controls the calculated odds ratio to be stored in the odds ratio storage unit 424b (S164).

Furthermore, the controller 422 transmits the result in step S163 to the web server 410 through the web server cooperation unit 423b. The web server 410 generates a short message containing the result through the SMS management unit 417 and then transmits the short message to the mobile communication terminal 10 of the attending physician of a corresponding patient, which is registered in advance, through the mobile communication network 100 (S165). In this case, the web server 410 may generate E-mail containing the result through the E-mail management unit 418 and transmit the E-mail to an E-mail account of the attending physician of a corresponding patient that is registered in advance.

A process of notifying the result through the short message or the E-mail in step S165 will now be described.

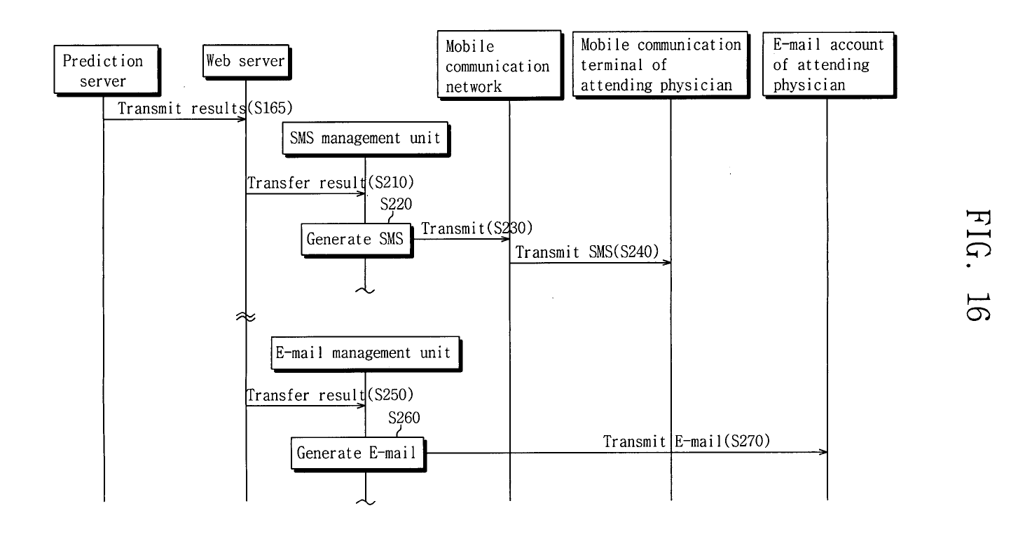

FIG. 16 is a flowchart for explaining a process of notifying the result based on the present invention.

Referring to FIG. 16, the prediction server 420 transmits the result to the web server 410 through the web server cooperation unit 423b (S165). Accordingly, the controller 411 of the web server 410 transfers the received result to the SMS management unit 417 (S210). The SMS management unit 417 that received the result generates a short message (SMS) containing the result (S220) and then transmits the short message to the mobile communication network 100 (S230). The mobile communication network 100 transfers the short message to the mobile communication terminal 10 of the attending physician (S240).

Meanwhile, the controller 411 of the web server 410 transfers the received result to the E-mail management unit 418 (S250). The E-mail management unit 418 that received the result generates E-mail containing the result (S260) and transmits the generated E-mail to an E-mail account of the attending physician through the Internet 200 (S270).

Accordingly, the attending physician of a patient can receive the result of liver cancer prediction in the form of the short message or E-mail through the mobile communication terminal. Therefore, the physician can consistently monitor the odds ratio of a patient and can take immediate action in case of emergency.

### INDUSTRIAL APPLICABILITY

As described above, based on the present invention, an estimation of the incidence rate for hepatocellular carcinoma and a relative risk of the incidence of liver cancer is calculated on an individual basis. It is thus possible to prevent the incidence of liver cancer by individual depending on the prediction of the incidence of liver cancer.

Furthermore, according to the present invention, hierarchical classification relating to a risk group for hepatocellular carcinoma is performed through an estimation of the incidence rate for hepatocellular carcinoma and a relative risk of the incidence of liver cancer, both of which are found on an individual basis. The present invention has an effect in that a tailored model for predicting the incidence of liver cancer is constructed.

In addition, based on the present invention, the attending physician of a patient can receive a result of liver cancer prediction in the form of a short message or E-mail through his or her mobile communication terminal. Therefore, the present invention has an effect in that the physician can consistently monitor the odds ratio of a patient and take immediate action in case of emergency.

(While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

1. A liver cancer prediction system for early detection, comprising:
a controller for controlling the entire operation of the system;
a display unit for displaying information and a graphic user interface depending on the operation of the system under the control of the controller;
an input unit for inputting initial set values, selecting a given menu based on the information displayed on the display unit and inputting information corresponding to the selected menu;
a plurality of databases for storing general information on a patient, information depending on an ultrasonic test performed, clinical information including information on findings upon a first registration of a patient and information on findings upon a diagnosis of liver cancer, and information on a risk group;
a regression counter for calculating a regression count which is an attributable ratio corresponding to each of risk factors based on the clinical information and risk group information stored in the database; and
an odds ratio measurement unit for measuring an odds ratio of the incidence of liver cancer by calculating risk probability of the incidence of liver cancer through a given operation process using the regression count calculated in the regression counter.

2. The liver cancer prediction system as claimed in claim 1, wherein the database comprises a patient information database for storing/managing information on a patient, an ultrasonic information database for storing/managing ultrasonic information, a clinical information database for storing/managing clinical information on the patient, and a risk group information database for storing/managing information on the risk group.

3. The liver cancer prediction system as claimed in claim 1, wherein the controller receives, from the database, registration information on a corresponding patient that is stored by default upon entry of information depending on the ultrasonic test, and then displays such information on an activation window automatically.

4. The liver cancer prediction system as claimed in claim 1, wherein clinical information includes parameters such as a diagnosis subject, hepatitis, a diagnosis basis, a case history, examination findings and an odds ratio.

5. The liver cancer prediction system as claimed in claim 1, wherein the odds ratio measurement unit uses three kinds of core risk factors including a diagnosis subject, the cause of hepatitis and AFP in order to calculate the risk probability.

6. The liver cancer prediction system as claimed in claim 1, wherein the odds ratio measurement unit uses extended risk factors where other control factors including the three kinds of the core risk factors are taken into consideration in order to calculate the risk probability.

7. The liver cancer prediction system as claimed in claim 1, wherein the odds ratio measurement unit uses risk factors consisting of hepatitis, liver cirrhosis, hepatitis furuncle, ALT, α-FP (feto protein), age, sex (man/female), tolerance level to alcohol, where drinking history is not known, probability for liver cancer, an odds ratio, risk probability and a risk group.

8. The liver cancer prediction system as claimed in claim 1, wherein the odds ratio measurement unit finds an attributable ratio (regression count) by using the logistic regression that corresponds to the risk factor and then calculates risk probability.

9. The liver cancer prediction system as claimed in claim 1, wherein the odds ratio measurement unit comprises an odds ratio storage unit for storing risk probability and an odds ratio of the incidence of liver cancer that are previously made.

10. The liver cancer prediction system as claimed in claim 1, wherein the controller comprises a calculation-selecting unit for selecting whether to calculate risk probability using a core risk factor or perform calculation using an extended risk factor.

11. The liver cancer prediction system as claimed in claim 1, wherein the controller comprises a trace search unit for searching a trace monitoring item from risk group-assigning materials that are previously stored in the database, in case where an extended risk factor is selected.

12. The liver cancer prediction system as claimed in claim 1, further comprising an SMS management unit for generating a short message containing information on the result measured in the odds ratio measurement unit, and then providing the short message to a mobile communication terminal of a patient's attending physician that is previously registered through a mobile communication network.

13. The liver cancer prediction system as claimed in claim 1, further comprising an E-mail management unit for generating E-mail containing information on the result measured in the odds ratio measurement unit and transmitting the generated E-mail to an E-mail account of a patient's attending physician that is previously registered.

14. A liver cancer prediction system for early detection, comprising:
a web server for providing a web service for predicting liver cancer to a user terminal through the Internet;
a database for storing general information on a patient, information depending on an ultrasonic operation performed, clinical information including information on findings upon a first registration of a patient and information on findings upon a diagnosis of liver cancer, and information on a risk group; and
a predicting server for performing liver cancer prediction based on information of the database.

15. The liver cancer prediction system as claimed in claim 14, wherein the web server comprises a controller for controlling the entire operation, a network connection unit for connection to the Internet, a web service unit for providing a web service for predicting liver cancer to the user terminal connected through the Internet, and a prediction server cooperation unit that cooperates with the prediction server to exchange data with the prediction server.

16. The liver cancer prediction system as claimed in claim 15, wherein the web server receives information on an odds ratio of the incidence of liver cancer calculated in the prediction server through the prediction server cooperation unit, and
the web server further comprises an SMS management unit for generating a short message containing received information on the odds ratio of the incidence of liver cancer and then providing the short message to a mobile communication terminal of a patient's attending physician that is previously registered through a mobile communication network.

17. The liver cancer prediction system as claimed in claim 15, wherein the web server receives information on an odds ratio of the incidence of liver cancer calculated in the prediction server through the prediction server cooperation unit, and
the web server further comprises an E-mail management unit for generating E-mail containing received information on the odds ratio of the incidence of liver cancer and providing the generated E-mail to an E-mail account of a patient's attending physician that is previously registered.

18. The liver cancer prediction system as claimed in claim 14, wherein the prediction server comprises:
a controller for controlling the entire operation of the system,
a display unit for displaying information depending on the operation of the system and a graphic user interface under the control of the controller,
an input unit for inputting initial set values, selecting a given menu according to information displayed on the display unit and inputting information corresponding to the selected menu,
a regression counter for calculating a regression count which is an attributable ratio corresponding to each of risk factors based on clinical information and risk group information stored in the database, and
an odds ratio measurement unit for measuring an odds ratio of the incidence of liver cancer by calculating risk probability of the incidence of liver cancer through a given operation process using the regression count calculated in the regression counter.

19. The liver cancer prediction system as claimed in claim 18, wherein the controller receives, from the database, registration information on a corresponding patient that is stored by default when information depending on the ultrasonic test is inputted, and then displays such information on an activation window automatically.

20. The liver cancer prediction system as claimed in claim 18, wherein clinical information includes parameters such as a diagnosis subject, hepatitis, a diagnosis basis, a case history, examination findings and an odds ratio.

21. The liver cancer prediction system as claimed in claim 18, wherein the odds ratio measurement unit uses three kinds of core risk factors including a diagnosis subject, the cause of hepatitis and AFP in order to calculate the risk probability.

22. The liver cancer prediction system as claimed in claim 18, wherein the odds ratio measurement unit uses extended risk factors where other control factors including the three kinds of the core risk factors are taken into consideration in order to calculate the risk probability.

23. The liver cancer prediction system as claimed in claim 18, wherein the odds ratio measurement unit uses extended risk factors consisting of hepatitis, liver cirrhosis, hepatitis furuncle, ALT, α-FP (feto protein), age, sex (man/female), tolerance level to alcohol, where drinking history is not known, probability for liver cancer, an odds ratio, risk probability and a risk group.

24. The liver cancer prediction system as claimed in claim 18, wherein the odds ratio measurement unit finds an attributable ratio (regression count) corresponding to the risk factor and then calculates risk probability using the logistic regression.

25. The liver cancer prediction system as claimed in claim 18, wherein the odds ratio measurement unit comprises an odds ratio storage unit for storing risk probability and an odds ratio of the incidence of liver cancer that are previously made.

26. The liver cancer prediction system as claimed in claim 18, wherein the controller comprises a calculation-selecting unit for selecting whether to calculate risk probability using a core risk factor or using an extended risk factor.

27. The liver cancer prediction system as claimed in claim 18, wherein the controller comprises a trace search unit for searching a trace monitoring item from risk group-assigning materials that are previously stored in the database, in case where an extended risk factor is selected.

28. The liver cancer prediction system as claimed in claim 14, wherein the database comprises a patient information database for storing/managing information on a patient, an ultrasonic information database for storing/managing ultrasonic information, a clinical information database for storing/managing clinical information on the patient, and a risk group information database for storing/managing information on the risk group.

29. A method of controlling liver cancer prediction system including a controller for controlling the entire operation of the system; a display unit for displaying information depending on the operation of the system and a graphic user interface under the control of the controller; an input unit for inputting initial set values, selecting a given menu according to information displayed on the display unit and inputting information corresponding to the selected menu; a plurality of databases for storing general information on a patient, information depending on an ultrasonic operation performed, clinical information including information on findings upon a first registration of a patient and information on findings upon a diagnosis of liver cancer, and information on a risk group; a regression counter for calculating a regression count which is an attributable ratio corresponding to each of risk factors based on clinical information and risk group information stored in the database; and an odds ratio measurement unit for measuring an odds ratio of the incidence of liver cancer by calculating risk probability of the incidence of liver cancer through a given operation process using the regression count calculated in the regression counter, comprising:
a patient information-managing step of displaying, on a display unit, a given menu wherein general information on a patient can be written, and storing information inputted through the input unit in the database;
an ultrasonic information-managing step of displaying, on the display unit, a corresponding menu wherein information depending on an ultrasonic test performed can be written, and storing information inputted through the input unit in the database;
a clinical information-managing step of displaying, on the display unit, a given menu wherein information on findings upon a first registration of a patient and information on finding upon a diagnosis of liver cancer can be written, and storing information inputted through the input unit in the database;
a risk group-assigning step of displaying, on the display unit, a menu of a given format wherein additional risk groups can be assigned after the clinical information-managing step, and storing s risk group assigned according to information inputted through the input unit in the database; and
an odds ratio measurement step of measuring an odds ratio of the incidence of liver cancer, by calculating probability of the incidence of liver cancer on the basis of clinical information stored in the clinical information-managing step and the risk group assigned in the risk group-assigning step.

30. The method as claimed in claim 29, wherein the ultrasonic information-managing step includes receiving, from the database, registration information on a corresponding patient that is stored by default when information depending on the ultrasonic test is inputted, and then displaying such information on the display unit.

31. The method as claimed in claim 29, wherein clinical information-managing step includes displaying clinical information including a diagnosis subject, hepatitis, a diagnosis basis, a case history, examination findings and an odds ratio, matching information selected or inputted through the input unit to respective factors of the clinical information and then storing the matched results in the database.

32. The method as claimed in claim 29, wherein the clinical information of the clinical information-managing step comprises detail parameters such as a serial number, a diagnosis subject, hepatitis, a diagnosis basis, a case history, examination findings, findings upon a diagnosis of liver cancer, a diagnosis method.

33. The method as claimed in claim 29, wherein the clinical information-managing step comprises:
first step of, after a serial number is inputted into a registration number inspection box and an enter key is then pressed, searching the serial number through a patient information table stored in the database;
second step of, as a result of the search in the first step, if the serial number is not present in the table, displaying a message indicating that the serial number does not exist on the display unit and if the serial number is present in the table, displaying information on a name, an attending physician and reason on the display unit, and determining whether corresponding clinical information is stored in the database; and
third step of, as a result of the determination in the second step, if corresponding clinical information does exist in the database, displaying contents stored in the database on the display unit, and if corresponding clinical information does not exist in the database, displaying a given guide message on the display unit.

34. The method as claimed in claim 29, wherein the odds ratio measurement step includes using three kinds of core risk factors including a diagnosis subject, the cause of hepatitis and AFP in order to calculate the risk probability.

35. The method as claimed in claim 29, wherein the odds ratio measurement step includes using an extended risk factor where other control factors including the three kinds of the core risk factors are taken into consideration in order to calculate the risk probability.

36. The method as claimed in claim 29, wherein the odds ratio measurement step includes using the risk factors consisting of hepatitis, liver cirrhosis, hepatitis furuncle, ALT, α-FP (feto protein), age, sex (man/female), tolerance level to alcohol, where drinking history is not known, probability for liver cancer, an odds ratio, risk probability and a risk group.

37. The method as claimed in claim 36, wherein the odds ratio measurement step includes finding an attributable ratio (regression count) corresponding to the risk factor and then calculates risk probability through logistic regression using the attributable ratio.

38. The method as claimed in claim 37, wherein the odds ratio measurement step comprises:
a first step of defining three core risk factors for setting a logistic regression model as numerical type parameters; and
a second step of inserting respective risk factor into a statistical prediction model depending in risk probability and an odds ratio of the incidence of liver cancer that are already made after being defined in the first step, thus displaying the odds ratio and a risk probability value based on the ratio on the display unit.

39. The method as claimed in claim 29, wherein the risk group-assigning step comprises:
first step of searching a serial number through a patient information table stored in the database after the serial number is inputted through the input unit and the enter key is pressed;
second step of, as a result of the search in the first step, if the serial number is not present in the table, displaying a message indicating that the serial number does not exist on the display unit, and if the serial number is present in the table, displaying information on a name and an attending physician and reason on the display unit and at the same time determining whether ‘risk group specification’ is stored in the database; and
third step of, as a result of the determination in the second step, if corresponding clinical information is stored in the database, displaying contents stored in the database on the display unit, and if corresponding clinical information is not stored in the database, displaying a given guide message on the display unit.

40. The method as claimed in claim 29, wherein the risk group-assigning step comprises a step of selecting whether to calculate risk probability using a core risk factor or using an extended factor.

41. The method as claimed in claim 40, wherein the risk group-assigning step comprises a step of searching a trace monitoring item from risk group-assigning materials that are previously stored in the database, in case where an extended risk factor is selected.

42. The method as claimed in claim 29, wherein liver cancer prediction system further comprises an SMS management unit for managing a short message, and
the method further comprises:
a step of generating a short message containing the result calculated in the odds ratio measurement step; and
a step of transmitting the short message to a mobile communication terminal of a patient's attending physician that is previously registered, through a mobile communication network.

43. The method as claimed in claim 29, wherein liver cancer prediction system further comprises an E-mail management unit for managing E-mail, and
the method further comprises:
a step of generating E-mail containing the result calculated in the odds ratio measurement step; and
a step of transmitting the generated E-mail to an E-mail account of a patient's attending physician that is previously registered.
